(19) United States
(12) Patent Application Publication
(10) Pub. No.: US 2025/0201357 A1
(43) Pub. Date: Jun. 19, 2025

(54) INFORMATION PROCESSING APPARATUS, INFORMATION PROCESSING SYSTEM, AND METHOD

(71) Applicants: Preferred Networks, Inc., Tokyo (JP); ENEOS Corporation, Tokyo (JP)

(72) Inventors: Wenwen LI, Tokyo (JP); So TAKAMOTO, Tokyo (JP); Nontawat CHAROENPHAKDEE, Tokyo (JP); Yuta TSUBOI, Tokyo (JP)

(73) Assignees: Preferred Networks, Inc., Tokyo (JP); ENEOS Corporation, Tokyo (JP)

(21) Appl. No.: 18/975,442

(22) Filed: Dec. 10, 2024

(30) Foreign Application Priority Data
Dec. 14, 2023 (JP) 2023-211218

(51) Int. Cl. G16C 20/70; G16C 20/20

(52) U.S. Cl. CPC G16C 20/70; G16C 20/20

(57) ABSTRACT

An information processing apparatus according to an embodiment includes at least one processor and at least one memory. The at least one processor determines, based on types of atoms to be analyzed by a second model, at least part of parameters of a first model having been trained with first data. The at least one processor generates, by using the at least part of parameters of the first model, the second model different from the first model. The second model is a model from which an analysis result of an atomic structure is output by inputting the atomic structure.

### CROSS-REFERENCE TO RELATED APPLICATIONS

This application is based upon and claims the benefit of priority from Japanese Patent Application No. 2023-211218, filed on Dec. 14, 2023; the entire contents of all of which are incorporated herein by reference.

### FIELD

Embodiments of the present disclosure relate generally to an information processing apparatus, an information processing system, and a method.

### BACKGROUND

As a conventional method of simulating atomic structures, there are coupled-cluster singles-and-doubles (CCSD), a simulation using a classical molecular dynamic potential (hereinafter referred to as a classical potential), a quantum chemical calculation such as a density functional theory (DFT), and a machine learning potential that has been learned by using DFT as correct data.

However, in the conventional simulation using the classical potential, a large number of atoms can be simulated, whereas the accuracy may be low.

In addition, in the CCSD, the DFT, and the machine learning potential noted above, the accuracy is high, whereas the number of atoms that can be simulated is limited. Therefore, there has been a demand for a method that enables a highly accurate simulation for a large number of atoms.

### SUMMARY

An information processing apparatus according to an embodiment includes at least one processor and at least one memory. The at least one processor determines, based on types of atoms to be analyzed by a second model, at least part of parameters of a first model having been trained with first data. The at least one processor generates, by using the at least part of parameters of the first model, the second model different from the first model. The second model is a model from which an analysis result of an atomic structure is output by inputting the atomic structure.

### BRIEF DESCRIPTION OF THE DRAWINGS

FIG. 1 is a block diagram illustrating an example of a configuration of an information processing system according to an embodiment;

FIG. 2 is a diagram illustrating an example of functional blocks of a processor according to the embodiment;

FIG. 3 is a diagram illustrating an example of a configuration of a first model according to the embodiment;

FIG. 4 is a diagram illustrating an example of a configuration of a second model according to the embodiment; and

FIG. 5 is a flowchart illustrating an example of a procedure of processing from training to evaluation performed by an information processing apparatus according to the embodiment.

### DETAILED DESCRIPTION

Hereinafter, an embodiment will be described in detail with reference to the drawings.

### Embodiment

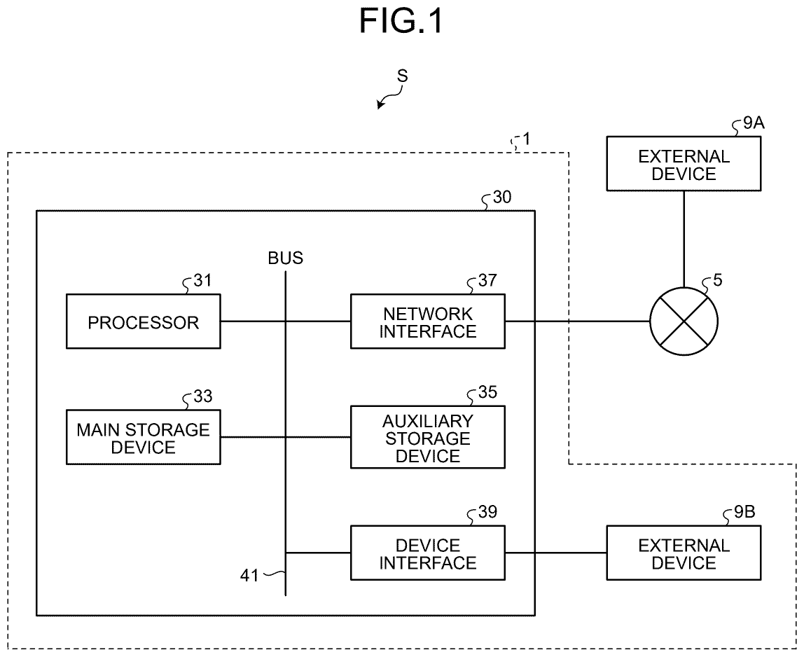

FIG. 1 is a block diagram illustrating an example of a configuration of an information processing system S according to the present embodiment. As illustrated in FIG. 1, the information processing system S of the present embodiment includes an information processing apparatus 1 and an external device 9A.

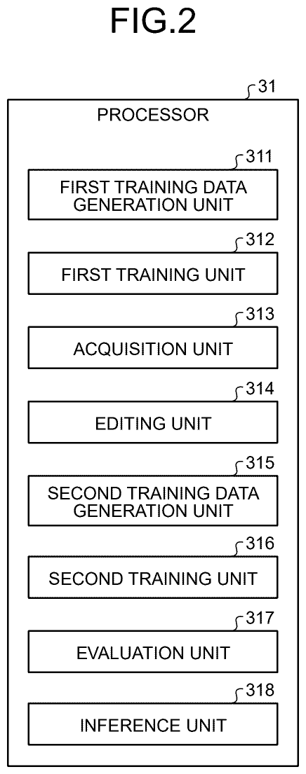

The information processing apparatus 1 is a computer that generates an artificial intelligence model serving to perform a molecular dynamics (MD) simulation and provides a simulation using the artificial intelligence model. The functions of the information processing apparatus 1 will be described in detail below with reference to FIG. 2 and the subsequent diagrams. The artificial intelligence model is a non-limiting example of a “model” in the present embodiment. The MD simulation is a non-limiting example of a “simulation” in the present embodiment. The information processing apparatus 1 is an example of a first information processing apparatus in the present disclosure.

The information processing apparatus 1 includes, for example, a computer 30 and an external device 9B that is connected to the computer 30 via a device interface 39. In one example, the computer 30 includes a processor 31, a main storage device (memory) 33, an auxiliary storage device (memory) 35, a network interface 37, and a device interface 39. The information processing apparatus 1 may be implemented by the computer 30 in which the processor 31, the main storage device 33, the auxiliary storage device 35, the network interface 37, and the device interface 39 are connected to each other via a bus 41.

In the computer 30 illustrated in FIG. 1, a single entity is provided per one element, but two or more entities may be provided per one element. One computer 30 is illustrated in FIG. 1. Alternatively, software may be installed in multiple computers, and these computers may perform the same part of processing of the software or different parts of processing of the software. In this case, the computers may communicate with each other, via the network interface 37 or the like, to perform processing in a distributed computing manner. In other words, the information processing apparatus 1 according to the present embodiment may be configured as a system that implements various functions to be described below by one or more computers executing commands stored in one or more storage devices. Additionally, information transmitted from a terminal may be processed by one or more computers provided on a cloud, and the processing result may be transmitted to a terminal such as a display device (display unit) corresponding to the external device 9B.

Various operations of the information processing apparatus 1 according to the present embodiment may be performed in parallel by using one or more processors or using multiple computers via a network. The various operations may be distributed to a plurality of arithmetic cores in the processor and performed in parallel processes. Part of or all the processing, means, and the like of the present disclosure may be performed by at least one of a processor or a storage device provided on a cloud communicable with the computer 30 via a network. In this manner, the various operations to be described below in the present embodiment may be performed by one or more computers in a manner of parallel computing.

The processor 31 may be an electronic circuit (a processing circuit, processing circuitry, a central processing unit (CPU), a graphics processing unit (GPU), a field programmable gate array (FPGA), an application specific integrated circuit (ASIC), or the like) including a control device and an arithmetic device of the computer 30. The processor 31 may be a semiconductor device or the like including a dedicated processing circuit. The processor 31 is not limited to an electronic circuit with electronic logic elements, and may be implemented by an optical circuit with optical logic elements. The processor 31 may have an arithmetic function based on quantum computing.

The processor 31 can perform arithmetic processing based on data and software (computer program) input from each device in the internal configuration of the computer 30, and output arithmetic results and a control signal to each device. The processor 31 may control each component constituting the computer 30 by executing an operating system (OS), an application, or the like of the computer 30.

The information processing apparatus 1 according to the present embodiment may be implemented by one or more processors 31. The processor 31 may refer to one or more electronic circuits disposed on one chip, or may refer to one or more electronic circuits disposed on two or more chips or two or more devices. When two or more electronic circuits are used, these electronic circuits may communicate with each other in a wired or wireless manner.

The main storage device 33 is a storage device that stores commands to be executed by the processor 31, various data, and the like, and information stored in the main storage device 33 is read by the processor 31. The auxiliary storage device 35 is a storage device other than the main storage device 33. Note that these storage devices mean optional electronic components capable of storing electronic information, and may be semiconductor memories. The semiconductor memory may be a volatile memory or a nonvolatile memory. The storage device for storing various data used in the information processing apparatus 1 according to the present embodiment may be implemented by the main storage device 33 or the auxiliary storage device 35, or may be implemented by a built-in memory built in the processor 31. In one example, the storage unit in the present embodiment is implemented by the main storage device 33 or the auxiliary storage device 35.

Multiple processors may be connected (coupled) to one storage device (memory). One processor 31 may be connected to one storage device (memory). Multiple storage devices (memories) may be connected (coupled) to one processor. In a case where the information processing apparatus 1 according to the present embodiment includes at least one storage device (memory) and multiple processors connected (coupled) to the at least one storage device (memory), at least one of the multiple processors may be connected (coupled) to the at least one storage device (memory). This configuration may be implemented by storage devices (memories) and processors 31 included in multiple computers. Moreover, a storage device (memory) may be integrated with the processor 31 (for example, a cache memory including an L1 cache and an L2 cache).

The network interface 37 is an interface for connection to the communication network 5 in a wireless or wired manner. As the network interface 37, an appropriate interface such as one conforming to an existing communication standard may be used. Information may be exchanged with the external device 9A connected via the communication network 5 by the network interface 37. Note that the communication network 5 may be any one of a wide area network (WAN), a local area network (LAN), a personal area network (PAN), or the like, or a combination thereof, as long as information is exchanged between the computer 30 and the external device 9A. Examples of the WAN include the Internet, examples of the LAN include IEEE802.11 and Ethernet (registered trademark), and examples of the PAN include Bluetooth (registered trademark) and near field communication (NFC).

The device interface 39 is an interface such as a universal serial bus (USB) directly connected to an output device such as a display device, an input device, and the external device 9B. Note that the output device may include a speaker or the like that outputs sound or the like.

The external device 9A is a device connected to the computer 30 via the communication network 5. The external device 9B is a device directly connected to the computer 30 via the device interface 39. Note that the information processing system S may include a plurality of external devices 9A and/or a plurality of external devices 9B. In this case, the information processing apparatus 1 may be communicably connected to each of the external devices 9A and/or the external devices 9B.

In one example, the external device 9A is a computer in which at least one processor, a main storage device, an auxiliary storage device, a network interface, and a device interface are connected to each other via a bus. The external device 9A is an example of another information processing apparatus or a second information processing apparatus in the present disclosure.

A trained artificial intelligence model according to the present embodiment has been installed in the information processing apparatus 1. The information processing apparatus 1 may be owned by a business operator that provides users with the use of the trained artificial intelligence model as a service. In this case, the external device 9A may be used by a user who uses the trained artificial intelligence model. The user can access the information processing apparatus 1 by operating the external device 9A, and perform a simulation using the trained artificial intelligence model stored in the information processing apparatus 1. The processor of the external device 9A may transmit information about the type of atom that the user desires to be analyzed and information about learning or analysis to the information processing apparatus 1.

Alternatively, the external device 9A or the external device 9B may be an input device (input unit). The input device is, for example, a device such as a camera, a microphone, a motion capture device, various sensors, a keyboard, a mouse, or a touch panel, which provides acquired information to the computer 30. The external device 9A or the external device 9B may be a device such as a personal computer, a tablet terminal, or a smartphone, which includes an input unit, a memory, and a processor.

Alternatively, the external device 9A or the external device 9B may be an output device (output unit). The output device may be, for example, a display device (display unit) such as a liquid crystal display (LCD), a cathode ray tube (CRT), a plasma display panel (PDP), or an organic electro luminescence (EL) panel, or may be a speaker that outputs sound or the like. The external device 9A or the external device 9B may be a device such as a personal computer, a tablet terminal, or a smartphone, which includes an output device, a memory, and a processor.

Alternatively, the external device 9A or the external device 9B may be a storage device (memory). For example, the external device 9A may be a network storage or the like, and the external device 9B may be a storage such as an HDD.

Alternatively, the external device 9A or the external device 9B may be a device having part of functions of the components of the information processing apparatus 1 in the present embodiment. The computer 30 may transmit or receive part of or all the processing results obtained by the external device 9A or the external device 9B.

FIG. 2 is a diagram illustrating an example of functional blocks of the processor 31 according to the present embodiment. The functions implemented by the processor 31 include, for example, a first training data generation unit 311, a first training unit 312, an acquisition unit 313, an editing unit 314, a second training data generation unit 315, a second training unit 316, an evaluation unit 317, and an inference unit 318. The functions implemented by the first training data generation unit 311, the first training unit 312, the acquisition unit 313, the editing unit 314, the second training data generation unit 315, the second training unit 316, the evaluation unit 317, and the inference unit 318 are stored as programs, for example, in the main storage device 33 or the auxiliary storage device 35. The processor 31 reads and performs the programs stored in the main storage device 33, the auxiliary storage device 35, or the like, thereby realizing the functions related to the first training data generation unit 311, the first training unit 312, the acquisition unit 313, the editing unit 314, the second training data generation unit 315, the second training unit 316, the evaluation unit 317, and the inference unit 318.

The first training data generation unit 311 generates training data (learning data) used for pre-training a first model. In the present embodiment, as one example, the first model to be pre-trained is a moment tensor potential (MTP). In the present embodiment, the first model before being pre-trained is also referred to as an initial model. The training data for pre-training is an example of the first data in the present disclosure.

In the present embodiment, the first training data generation unit 311 generates training data for pre-training by using a trained model. The model used for generating training data for pre-training is a model different from the first model and a second model to be described below. As the model for generating training data, for example, a neural network potential (NNP) trained with a density functional theory (DFT) as correct data can be adopted. The trained NNP used for generating training data for pre-training is, for example, a general-purpose neural network potential that can handle optional atomic structures. The trained NNP includes, for example, an input layer, one or more graph convolution layers, and an output layer. The trained NNP is an example of a third model in the present disclosure. The training data for pre-training is an example of the first data in the present disclosure.

The processing performed by the trained NNP is faster than the processing by the DFT. Therefore, by using the trained NNP having been trained in advance with the DFT as correct data for creating training data, the time required for generating training data can be shortened as compared with that in a case where the DFT is directly used.

The training data for pre-training includes atomic structures and analysis results of the atomic structures. The atomic structures include plural types (elements) of atoms and position information (atomic coordinates). The analysis results of the atomic structures include information about at least energies or forces of the atomic structures. In addition, the analysis results may further include indexes for evaluating stresses, densities, or other physical property values of the atomic structures. Examples of the indexes for evaluating the physical property values of the atomic structures include elastic modules.

The training data may further include information about a cell, a periodic boundary condition, or a type of simulation. The cell is a box in which an environment for executing a simulation is defined, and is set for each type of simulation. The type of simulation is also called a use case, and is defined by, for example, a precondition for the analysis of the atomic structure. Specifically, the type of simulation is a temperature, a surface structure of a substance constituted by an atomic structure, or the like, which is a precondition for simulation. In one example, the type of simulation is defined as “a simulation as to how the structure changes when the temperature is raised to xx degrees from given initial atomic coordinates” or the like.

The first training unit 312 generates a trained first model by training the model with the first data. More specifically, the first training unit 312 generates the trained first model by training the initial model with the training data for pre-training generated by the first training data generation unit 311.

The acquisition unit 313 acquires, from the external device 9A, information to be used for generating a second model described below. More specifically, the acquisition unit 313 acquires, from the external device 9A, information including types of atoms to be analyzed. In the present embodiment, the types of atoms may include types of elements to be analyzed and differences between environments in which the atoms to be analyzed are placed. In one example, when the environments in which the atoms are placed are different, these atoms may be handled as different types of atoms. The atoms or elements to be analyzed are, for example, atoms or elements included atomic structures that the user desires to analyze. The atoms or elements to be analyzed are objects to be analyzed by a second model generated based on the first model. The second model will be described below. Note that the source from which types of atoms to be analyzed are acquired is not limited to the external device 9A. For example, the acquisition unit 313 may acquire types of atoms to be analyzed from the external device 9B. Alternatively, the types of atoms to be analyzed may be stored in advance in the auxiliary storage device 35. The acquisition unit 313 may acquire information about atomic structures to be analyzed, for example, molecular structures, crystal structures, surface structures, combinations thereof, or the like, from the external device 9A or another acquisition source. The acquisition unit 313 may acquire information about phenomena or physical properties to be analyzed, for example, elastic modules, chemical reactions, densities, vibration characteristics, diffusions, or the like, or may acquire environments to be analyzed, for example, temperatures, pressures, or the like, from the external device 9A or another acquisition source.

The editing unit 314 generates a second model different from the first model by using at least part of parameters of the trained first model that has been trained with the training data for pre-training. The second model is generated based on the first model, so that the first model and the second model are artificial intelligence models of the same type. Specifically, in the present embodiment, the first model and the second model are each an MTP model.

The processing load of the MTP model is relatively low because there is no processing corresponding to multiple layers in contrast to another neural network such as a graph neural network (GNN). Therefore, in the present embodiment, by configuring the first model and the second model as MTP models, the processing can be speeded up.

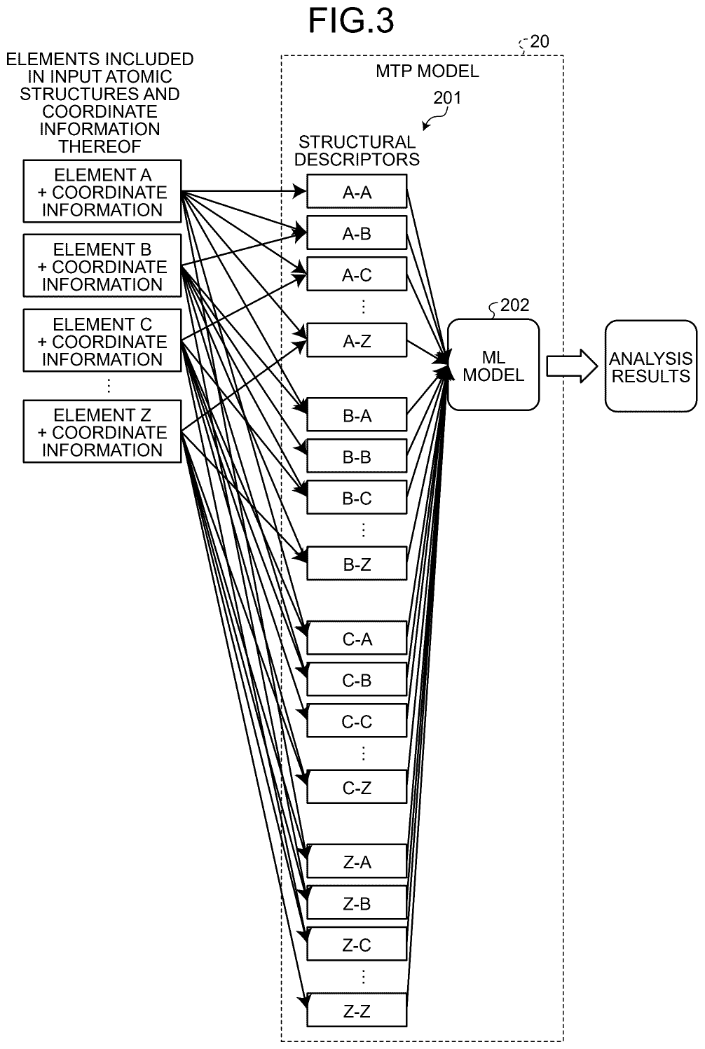

FIG. 3 is a diagram illustrating an example of a configuration of a first model 20 according to the present embodiment. The first model 20 is a model from which analysis results of atomic structures are output by receiving an input of these atomic structures.

As described above, the first model 20 is an MTP model. Thus, the first model 20 includes a plurality of structural descriptors 201 and a machine learning (ML) model 202. The structural descriptors 201 correspond to elements to be input to the first model 20. The structural descriptors 201 are examples of parameters in the present disclosure. The ML model 202 of the trained first model 20 also includes parameters based on the first training data and hyper-parameters that can be set by the user. The parameters based on the first training data included in the ML model 202 are, for example, regression coefficients in a case where the ML model is a linear regression model. Various parameters included in the ML model 202 may also be examples of parameters in the present disclosure.

The structural descriptors 201 are parameters determined on a rule basis for each type of atom. More specifically, the structural descriptors 201 are parameters determined on a rule basis for each type of element (chemical element). The parameters are determined for each pair of two elements. In one example, the data input to the first model 20 in an inference process is atomic structures, and more specifically, is information representing multiple types (elements) of atoms and position information (coordinate information or the like) these atoms. One structural descriptor 201 receives an input of two elements and position information of the two elements for each pair of two types of elements included in the input atomic structure.

The ML model 202 is provided at a stage subsequent to the structural descriptors 201 so as to perform processing based on data acquired from the structural descriptors 20 and output analysis results of the atomic structures. Specifically, the ML model 202 of the present embodiment is a regression model based on a polynomial basis function. The ML model 202 is updated by training.

The editing unit 314 of the present embodiment generates a second model that includes part of the structural descriptors 201 in the trained first model 20 and the ML model 202. The structural descriptors 201 included in the second model correspond to the elements to be analyzed that have been acquired by the acquisition unit 313. In other words, the editing unit 314 determines structural descriptors 201 to be included in the second model, based on the types of atoms to be analyzed that have been acquired by the acquisition unit 313.

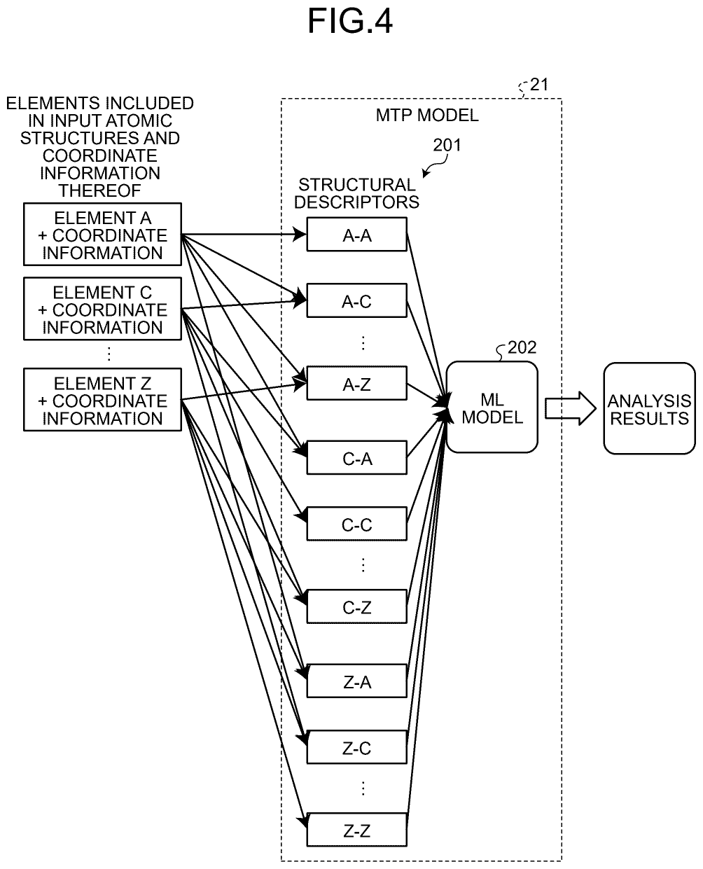

FIG. 4 is a diagram illustrating an example of a configuration of a second model 21 according to the present embodiment. The second model 21 is a model from which analysis results of atomic structures are output by receiving an input of these atomic structures.

In the present embodiment, as one example, it is assumed that the element B is not included in the elements to be analyzed that have been acquired by the acquisition unit 313. In this case, the editing unit 314 excludes structural descriptors 201 corresponding to the element B from the structural descriptors 201 included in the trained first model 20. Then, the editing unit 314 generates a second model 21 that includes the structural descriptors 201 corresponding to the elements to be analyzed and the ML model 202. In the present embodiment, it is not necessary to change the ML model 202 at the time of generating the second model 21. The second model 21 is in a non-trained state at the time of generation. Thus, various parameters of the ML model 202 are similar to parameters of the trained first model 20.

The second model 21 contains structural descriptors 201 extracted by the editing unit 314 from among the structural descriptors 201 in the trained first model 20. Thus, the number of structural descriptors 201 in the second model 21 is smaller than the number of structural descriptors 201 in the first model 20. Therefore, in the present embodiment, the number of types of atoms that can be analyzed by the first model 20 is larger than the number of types of atoms that can be analyzed by the second model 21.

In the inference process of the MTP model, processing should be performed also on a structural descriptor 201 that corresponds to an element not included in the input atomic structure. Therefore, the processing speed can be improved by excluding unnecessary structural descriptors 201 in advance in the second model 21.

Returning to FIG. 2, the second training data generation unit 315 generates training data used for fine-tuning the second model 21. In the present embodiment, the second training data generation unit 315 generates the training data for fine-tuning by using, for example, an NNP trained with the DFT as correct data. The trained NNP used for generating the training data for fine-tuning may be the same model as the trained NNP used for generating training data for pre-training described above. The trained NNP is an example of a third model in the present disclosure. Note that the model used for generating training data for pre-training and the model used for generating training data for fine-tuning may be different models. The training data for fine-tuning is an example of second data in the present disclosure.

The second training data generation unit 315 in the present embodiment generates second data by using a trained NNP that has been trained with the DFT as correct data. The second data includes: atomic structures including elements to be analyzed that have been acquired by the acquisition unit 313, and analysis results of the atomic structures. The atomic structures in the second data do not include elements other than the elements to be analyzed. The second data may further include information about a cell, a periodic boundary condition, or a type of simulation.

The second training unit 316 generates a trained second model 21 by training a non-trained second model 21 with the second data. The trained second model 21 is a model from which analysis results of atomic structures are output by inputting these atomic structures. The second training unit 316 stores the generated trained second model 21, for example, in the auxiliary storage device 35.

More specifically, the second training unit 316 performs fine-tuning on the non-trained second model 21 so as to match with the elements to be analyzed. By performing the fine-tuning, the ML model 202 included in the non-trained second model 21 is trained, and the parameters of the ML model 202 are updated.

In the pre-training described above, for training the first model 20 so as to generally handle optional atomic structures, it is preferable that the training data includes various atomic structures, not limited to atomic structures including elements to be analyzed that are assumed at the time of actual use. Therefore, the number of types of atomic structures included in the first data that is training data for pre-training is larger than the number of types of atomic structures included in the second data that is training data for fine-tuning.

On the other hand, in the fine-tuning, the second model 21 is trained specifically for elements to be analyzed that are assumed at the time of actual use. Thus, although versatility is lowered, the processing speed for the elements to be analyzed can be improved. Therefore, in the inference process related to the target elements, the second model 21 can simulate a larger number of atoms in one inference process than the first model 20.

The second training unit 316 may perform the training up to a predetermined number of epochs. Alternatively, the second training unit 316 may divide the training data (second data) into learning data and validation data, observe a change in loss (magnitude of error) for each of the learning data and the validation data during the training, and continue the training until determining that the loss has completely decreased. Note that the criterion for completing the training may be designated by, for example, the user via the external device 9A. Alternatively, the criterion for completing the training may be stored in advance in the auxiliary storage device 35 or the like of the information processing apparatus 1, based on the accuracy and the processing speed required for the trained second model 21. In one example, the user may perform an operation changing the learning rate in the middle of training, an operation of changing the number of epochs according to the model capacity, or the like. The model capacity is a size of a structure of the MTP model, and for example, the larger the model capacity, the larger the number of atoms for which the trained second model 21 analyzes relationships. The larger the model capacity, the higher the accuracy of the analysis process, but the slower the processing speed.

Note that, even in the pre-training by the first training data generation unit 311 described above, the completion of the training may be determined based on the number of epochs or based on losses of training data and validation data during learning, or an operation of setting or changing the criterion for terminating the training may be received from the user. The pre-training and the fine-tuning may be different in the criterion for completing the training.

The evaluation unit 317 compares the analysis results output from the second model 21 with verification data, and outputs a comparison result as a numerical value.

The verification data is generated by a method different from a method using the first model 20 and a method using the second model 21. More specifically, the verification data is analysis results obtained by a method using the third model that has generated at least one of the first data or the second data. For example, in a case where the first data that is training data for pre-training and the second data that is training data for fine-tuning are generated by trained NNPs that have been trained with the DFT as correct data, the evaluation unit 317 may generate the verification data by using such a trained NNP.

The verification data includes, for example, atomic structures and analysis results of the atomic structures, similarly to the training data. As described above, the processing speed of the trained NNP is faster than that of the DFT. Thus, by using the trained NNP that has been trained in advance with the DFT as correct data for creating verification data, the time required for generating verification data can be shortened as compared with that in a case where the DFT is directly used. Note that the evaluation unit 317 may use some pieces of the generated first data or second data as the verification data. In addition, the verification data may further include a cell, a periodic boundary condition, or a type of simulation.

The evaluation unit 317 inputs atomic structures of verification data to the trained second model 21. Then, the evaluation unit 317 compares analysis results of the atomic structures output from the trained second model 21 with the analysis results of the atomic structures of the verification data. As data input at the time of verification, information about a cell, a periodic boundary condition, or a type of simulation may be further input to the trained second model 21.

The evaluation unit 317 evaluates the analysis results of the trained second model 21 on the assumption that the analysis results of the atomic structures of the verification data are correct. Specifically, the evaluation unit 317 compares the analysis results of the atomic structures output from the trained second model 21 with the analysis results of the atomic structures of the verification data, and evaluates whether or not the two results match with each other.

In addition, the evaluation unit 317 evaluates whether or not a simulation process of the trained second model 21 fails. The failure in the simulation process means, for example, that the simulation process of the trained second model 21 to which the input data for verification has been input does not normally end, and thereby no analysis result can be obtained.

The numerical value representing the comparison result is, for example, a success rate of the simulation process, a mean absolute error (MAE) of the analysis results, a time required for the simulation process, or the like. The success rate of the simulation process is a ratio of: the number of simulation processes that have normally ended, to the total number of simulation processes. The MAE of the analysis results is an average of absolute values of differences between the analysis results of the atomic structures of the verification data, which are true values, and analysis results of the trained second model 21. For example, in a case where the analysis results are densities of the atomic structures, the evaluation unit 317 calculates an average of absolute values of differences between density values of the training data generated by using the trained NNP and densities output from the trained second model 21. Note that the indexes used by the evaluation unit 317 for the evaluation of the trained second model 21 are not limited thereto, and optional indexes can be adopted.

A method of outputting the analysis results obtained by the evaluation unit 317 is not particularly limited. The analysis results may be displayed on a display, may be stored as data in various storage devices, or may be transmitted to the external device 9A or the external device 9B. In one example, the evaluation unit 317 outputs, on a display device or the like, the analysis results obtained by the trained second model 21 and the analysis results of the atomic structure of the verification data so as to arrange them side by side for each evaluation index.

The evaluation unit 317 may evaluate the trained second model 21 for each type of simulation. In this case, the type of simulation performed by the trained second model 21 and the type of simulation performed by the trained NNP at the time of generating the verification data are the same.

In a case where the evaluation of the trained second model 21 by the evaluation unit 317 does not meet a predetermined level, fine-tuning may be additionally performed on an atomic structure or a type of simulation, each resulting in a low evaluation. For example, the second training data generation unit 315 may additionally generate training data for fine-tuning (second data) based on the evaluation by the evaluation unit 317. The second training unit 316 may further fine-tune the trained second model 21 based on the additionally generated second data. In other words, the evaluation unit 317, the second training data generation unit 315, and the second training unit 316 may perform active learning of the second model 21.

The inference unit 318 performs a simulation process by the trained second model 21. More specifically, by inputting atomic structures to the fine-tuned second model 21, the inference unit 318 obtains analysis results of the atomic structures output by the fine-tuned second model 21. In one example, in response to receiving a user's operation input through the external device 9A, the inference unit 318 performs the simulation process. The atomic structure to be analyzed in the simulation process may be acquired from, for example, the external device 9A or the external device 9B. Alternatively, the atomic structure to be analyzed in the simulation process may be stored in the auxiliary storage device 35 of the information processing apparatus 1.

Note that, in a case where the user operates the external device 9A to access the information processing apparatus 1 and perform a simulation by using the trained second model 21 stored in the information processing apparatus 1, dynamics calculations using energies, forces, or the like output as analysis results from the trained second model 21 are performed by using calculation resources on the external device 9A side.

As data input in the simulation process, information about a cell, a periodic boundary condition, or a type of simulation may be further input to the trained second model 21.

Note that, in a case where the external device 9A is a computer used by a user, the structure and parameters of the second model 21 may not be viewable by the user who uses the external device 9A. The inference unit 318 may display, on the display of the external device 9A via a browser or another application, a screen including an input field in which the user can input an atomic structure to be analyzed and other input data and a button for executing a simulation process. In this case, even though the user cannot directly refer to the second model 21, a simulation can be performed by the trained second model 21. For exchange of data between the user-viewable application and the trained second model 21, a technology such as socket communication or shared memory can be employed, for example. By exchanging data through the socket communication or the shared memory, high-speed communication can be performed even though the user does not directly access the trained second model 21. Also, the structure and parameters of the first model 20 may not be viewable by the user who uses the external device 9A. In addition, the first model 20 and the non-fine-tuned second model 21 may not be accessible and usable by the user.

The inference unit 318 outputs analysis results of the simulation process to, for example, the external device 9A or the external device 9B. A method of outputting the analysis results obtained by the inference unit 318 is not particularly limited, and the analysis results may be displayed on a display, or may be stored as data in various storage devices.

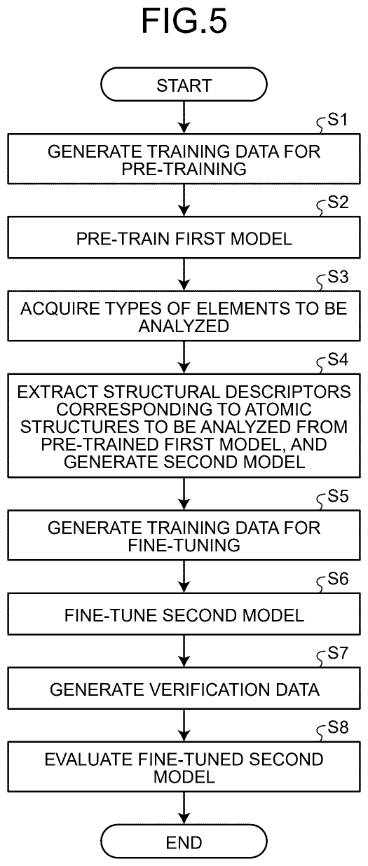

Next, a procedure of processing from training to evaluation performed by the information processing apparatus 1 of the present embodiment configured as described above will be described. FIG. 5 is a flowchart illustrating an example of a procedure of processing from training to evaluation performed by the information processing apparatus 1 according to the present embodiment.

First, the first training data generation unit 311 generates training data (first data) used for pre-training (S1). Specifically, the first training data generation unit 311 inputs atomic structures to an NNP that has been trained with DFT as correct data, and obtains analysis results of the atomic structures output from the trained NNP. In generation of the training data for pre-training, atomic structures including various elements, not limited to elements to be analyzed, may be input to the NNP. The first training data generation unit 311 generates plural sets of training data, each set consisting of the input atomic structures and the output analysis results of the input atomic structures. The first training data generation unit 311 may further input information about a cell, a periodic boundary condition, or a type of simulation to the trained NNP, and this information may be included in the training data.

The first training unit 312 pre-trains the first model 20 with the first data (S2).

The acquisition unit 313 acquires types of elements to be analyzed, from the external device 9A (S3).

The editing unit 314 extracts, from the pre-trained first model 20, structural descriptors 201 corresponding to the atomic structures to be analyzed, which have been acquired by the acquisition unit 313, and generates a second model 21 including the extracted structural descriptors 201 and an ML model 202 (S4).

The second training data generation unit 315 generates training data (second data) used for fine-tuning, based on the types of elements to be analyzed that have been acquired by the acquisition unit 313 (S5). Specifically, the second training data generation unit 315 inputs atomic structures including only elements to be analyzed to an NNP trained with DFT as correct data, and obtains analysis results of the atomic structures output from the trained NNP. The second training data generation unit 315 generates plural sets of training data, each set consisting of the input atomic structures and the output analysis results of the atomic structures. The second training data generation unit 315 may further input information about a cell, a periodic boundary condition, or a type of simulation to the trained NNP, and this information may be included in the training data. The trained NNP used for generating training data for fine-tuning may be the same as the model used in the process of generating training data for pre-training in step S1 described above.

The second training unit 316 fine-tunes the second model 21 by using the second data (S6).

Then, the evaluation unit 317 generates verification data by a trained NNP having been trained with DFT as correct data (S7), and evaluates the fine-tuned second model 21 by using the verification data (S8). Specifically, the evaluation unit 317 inputs the atomic structures input to the trained NNP at the time of generating verification data to the fine-tuned second model 21, and compares analysis results output by the fine-tuned second model 21 with the verification data. In addition, the evaluation unit 317 evaluates whether or not a simulation process of the trained second model 21 fails. The evaluation unit 317 outputs an evaluation result, and the processing of this flowchart ends.

When the evaluation of the trained second model 21 does not meet a predetermined level, the processing of S5 to S7 may be repeatedly performed until the evaluation reaches the predetermined level.

Moreover, in FIG. 5, the processes from the generation of the training data for pre-training to the evaluation are illustrated consecutively, whereas there may be a time interval between the processes. In one example, after the processes up to pre-training have been performed in advance, fine-tuning may be performed at optional timing according to requests of individual users.

As described above, the information processing apparatus 1 of the present embodiment generates the second model 21 different from the first model 20 by using at least part of parameters of the first model 20 having been trained with the first data. Specifically, the second model 21 of the present embodiment has at least part of the structural descriptors 201 of the trained first model 20. Therefore, according to the information processing apparatus 1 of the present embodiment, by generating the second model 21 from the trained first model 20, the speed of the simulation process can be enhanced, enabling a highly accurate simulation for a large number of atoms.

The information processing apparatus 1 of the present embodiment determines parameters to be included in the second model 21, from among the parameters, e.g., the structural descriptors 201, of the trained first model 20, based on the types of elements to be analyzed by the second model 21. Therefore, according to the information processing apparatus 1 of the present embodiment, it is possible to adopt parameters suitable for the types of elements to be analyzed by the second model 21. Specifically, the information processing apparatus 1 of the present embodiment extracts some structural descriptors 201 from among the structural descriptors 201 included in the first model 20 to generate a second model 21. The structural descriptors 201 to be extracted are determined based on the types of elements to be analyzed by the second model 21. Therefore, the information processing apparatus 1 of the present embodiment can improve the speed of the simulation process by removing structural descriptors 201 related to elements not to be analyzed.

The information processing apparatus 1 of the present embodiment trains the second model 21 with the second data. Specifically, the information processing apparatus 1 of the present embodiment fine-tunes the second model 21 generated by the pre-trained first model 20. Therefore, according to the information processing apparatus 1 of the present embodiment, it is possible to improve the accuracy of the simulation with respect to the processing related to the second data.

The first data and the second data of the present embodiment are data generated by using a trained third model different from the first model 20, specifically, an NNP trained with DFT as correct data. The processing by the trained NNP is faster than the processing by the DFT. For this reason, by using the trained NNP that has been trained in advance with the DFT as correct data for creating first data and second data, the time required for generating first data and second data can be shortened as compared with that in a case where the DFT is directly used.

The first data and the second data of the present embodiment include atomic structures and analysis results of the atomic structures, and the number of types of atomic structures included in the first data is larger than the number of types of atomic structures included in the second data. In the pre-training, in order to train the first model 20 to generally handle optional atomic structures, it is preferable that the training data includes various atomic structures, not limited to atomic structures including elements to be analyzed that are assumed at the time of actual use. Therefore, as in the present embodiment, since the number of types of atomic structures included in the first data that is training data for pre-training is larger than the number of types of atomic structures included in the second data that is training data for fine-tuning, the first model 20 can be pre-trained to handle various atomic structures.

The information processing apparatus 1 of the present embodiment determines structural descriptors 201 to be included in the second model 21, from among the structural descriptors 201 in the first model 20, based on information acquired from the external device 9A. The information acquired from the external device 9A includes types of elements to be analyzed by the second model 21. In one example, by inputting, by the user, elements included in atomic structures desired to be analyzed from the external device 9A, the information processing apparatus 1 of the present embodiment can configure the structural descriptors 201 of the second model 21 so as to match with the user's need.

The first model 20 and the second model 21 of the present embodiment are models of the same type. Therefore, according to the information processing apparatus 1 of the present embodiment, the parameters of the ML model 202 of the first model 20 generated by the pre-training can be passed on to the second model 21.

In the present embodiment, the number of structural descriptors 201 included in the second model 21 is smaller than the number of structural descriptors 201 included in the first model 20. The smaller the number of structural descriptors 201, the smaller the processing amount. Therefore, the information processing apparatus 1 of the present embodiment can improve the speed of the simulation process of the second model 21.

The first model 20 and the second model 21 of the present embodiment are MTP models. As described above, the processing load of the MTP model is relatively low because there is no processing corresponding to multiple layers in contrast to another neural network such as a graph neural network (GNN). Therefore, the information processing apparatus 1 of the present embodiment can speed up the processing by configuring the first model 20 and the second model 21 as MTP models.

In the present embodiment, the analysis results of the atomic structures output from the second model 21 include information about at least energies or forces of the atomic structures. The analysis of the atomic structures has a high processing load, and there is a need for high-speed processing in order to analyze a larger number of atoms. In the information processing apparatus 1 of the present embodiment, it is possible to provide an MD simulation for the atomic structures by the fine-tuned second model 21 in response to such a need.

The information processing apparatus 1 of the present embodiment compares the analysis results output from the second model 21 with verification data generated by means for generation (that is, by a model) different from the first model 20 and the second model 21, and outputs a comparison result as a numerical value. Therefore, according to the information processing apparatus 1 of the present embodiment, it is possible to objectively determine whether or not fine-tuning of the second model 21 is sufficient. This makes it possible to takes measures such as additional training for the second model 21 according to an evaluation result.

In the present embodiment, the verification data is analysis results obtained by a third model that has generated at least one of the first data or the second data, that is, a trained NNP trained with DFT as correct data. Therefore, according to the information processing apparatus 1 of the present embodiment, it is possible to shorten the time required for generating verification data by using the trained NNP.

### Modification

In the above-described embodiment, the processes described as the functions of the information processing apparatus 1 may each be performed by the same apparatus or may be performed by different apparatuses. For example, the training of the first model 20, the training of the second model 21, the generation of the training data for pre-training, the generation of the training data for fine-tuning, the generation of the verification data, and the inference process using the trained second model 21 may be performed by the same apparatus as in the above-described embodiment, or may be performed by different apparatuses. Specifically, in the above-described embodiment, the information processing apparatus 1 generates training data for pre-training the first model 20 and performs pre-training, whereas these processes may not be performed in the information processing apparatus 1. In one example, the information processing apparatus 1 may use the pre-trained first model 20 by acquiring from another information processing apparatus.

In addition, in the above-described embodiment, it has been described as one example that the NNP trained with the DFT as correct data generates training data for pre-training and verification data. However, the training data for pre-training and the verification data may be generated by the DFT.

In addition, in the above-described embodiment, the verification data used for evaluating the second model 21 is generated by the trained NNP that has been trained with the DFT as correct data. However, the verification data generation method is not limited thereto. For example, the information processing apparatus 1 may acquire data on energies, forces, stresses, densities, elastic values, or the like of atomic structures through experiment, and use the acquired data as verification data. By using the data obtained through experiment as the verification data, it is possible to eliminate the effect of the accuracy of the model for generating verification data on evaluation results.

In addition, in the above-described embodiment, the various parameters of the ML model 202 of the non-trained second model 21 are similar to the various parameters of the ML model 202 of the trained first model 20. However, the parameters may be different between the ML model 202 of the non-trained second model 21 and the ML model 202 of the trained first model 20. In other words, the editing unit 314 of the information processing apparatus 1 may change the parameters of the ML model 202 in addition to the structural descriptors 201 of the pre-trained first model 20.

In addition, in the above-described embodiment, the ML model 202 included in the first model 20 and the second model 21 is a regression model based on a polynomial basis function. However, the ML model 202 is not limited thereto, and optional models can be adopted. In one example, the ML model 202 is a neural network (NN).

In the above-described embodiment, the first model 20 and the second model 21 are configured by MTP models. However, other models may be adopted. For example, GNNs can be adopted as the first model 20 and the second model 21. In this case, the information processing apparatus 1 may perform atom type embedding at a stage before the GNNs. As the first model 20 and the second model 21, neural network potentials expressing an environment of each atom with a symmetry function, for example, Behler-Parrinello type neural networks or Gaussian approximation potentials, may be adopted.

In addition, in the above-described embodiment, the trained second model 21 is stored in the auxiliary storage device 35 of the information processing apparatus 1. However, the trained second model 21 may be stored in an information processing apparatus owned by the user. For example, in a case where the external device 9A is a user's computer that uses the trained second model 21, the information processing apparatus 1 may transmit the trained second model 21 to the external device 9A. Note that, when the inference (analysis process) is performed by using the trained second model 21 on the information processing apparatus 1 side, the process can be performed at a higher speed than when inference is performed by the external device 9A using the trained second model 21.

In addition, in the above-described embodiment, the case where one second model 21 is generated from one trained first model 20 has been described. However, different second models 21 may be generated from one trained first model 20. The different second models 21 may be different in atomic structure to be analyzed or type of simulation.

The difference between the different second models may be a difference in fine-tuning data, a difference in structural descriptor 201, or a difference in ML model 202.

For example, the editing unit 314 of the information processing apparatus 1 may generate plural second models 21 that are different in structural descriptor 201 for each atomic structure to be analyzed.

In addition, different trained second models 21 may be generated from the same non-trained second model 21. For example, the second training data generation unit 315 may generate plural pieces fine-tuning data (second data) including different atomic structures. In this case, the second training unit 316 may generate different trained second models 21 by using the plural pieces of fine-tuning data including different atomic structures.

Note that, in a case where plural trained second models 21 are generated for each atomic structure to be analyzed, when the inference unit 318 performs an inference process, a model name for specifying the trained second model 21 to be performed is also included in input data for a simulation process. The model name for specifying the trained second model 21 to be performed may be input, for example, by the user from the external device 9A or the external device 9B.

A part or the whole of the device (the information processing apparatus 1, the external device 9A, 9b) in the above-described embodiments may be configured by hardware, or may be configured by information processing of software (a program) performed by a CPU, a GPU, or the like. In the case where the embodiment is configured by the information processing of software, software implementing at least a part of the functions of each device in the above-described embodiment may be stored in a non-temporary storage medium (a non-temporary computer-readable medium) such as a compact disc-read only memory (CD-ROM) or a universal serial bus (USB) memory, and may be read into a computer to perform the information processing of software. The software may be downloaded via a communication network. Further, all or a part of the processing of software may be implemented in a circuit such as an application specific integrated circuit (ASIC) or a field programmable gate array (FPGA), so that information processing by the software may be performed by hardware.

The storage medium storing software may be a detachable storage medium such as an optical disk or a fixed storage medium such as a hard disk drive or a memory. Additionally, the storage medium may be provided inside the computer (a main storage device, an auxiliary storage device, and the like) or outside the computer.

In the present specification (including the claims), if the expression “at least one of a, b, and c” or “at least one of a, b, or c” is used (including similar expressions), any one of a, b, c, a-b, a-c, b-c, or a-b-c is included. Multiple instances may also be included in any of the elements, such as a-a, a-b-b, and a-a-b-b-c-c. Further, the addition of another element other than the listed elements (i.e., a, b, and c), such as adding d as a-b-c-d, is included.

In the present specification (including the claims), if the expression such as “in response to data being input”, “using data”, “based on data”, “according to data”, or “in accordance with data” (including similar expressions) is used, unless otherwise noted, a case in which the data itself is used and a case in which data obtained by processing the data (e.g., data obtained by adding noise, normalized data, a feature amount extracted from the data, and intermediate representation of the data) is used are included. If it is described that any result can be obtained “in response to data being input”, “using data”, “based on data”, “according to data”, or “in accordance with data” (including similar expressions), unless otherwise noted, a case in which the result is obtained based on only the data is included, and a case in which the result is obtained affected by another data other than the data, factors, conditions, and/or states may be included. If it is described that “data is output” (including similar expressions), unless otherwise noted, a case in which the data itself is used as an output is included, and a case in which data obtained by processing the data in some way (e.g., data obtained by adding noise, normalized data, a feature amount extracted from the data, and intermediate representation of the data) is used as an output is included.

In the present specification (including the claims), if the terms “connected” and “coupled” are used, the terms are intended as non-limiting terms that include any of direct, indirect, electrically, communicatively, operatively, and physically connected/coupled. Such terms should be interpreted according to a context in which the terms are used, but a connected/coupled form that is not intentionally or naturally excluded should be interpreted as being included in the terms without being limited.

In the present specification (including the claims), if the expression “A configured to B” is used, a case in which a physical structure of the element A has a configuration that can perform the operation B, and a permanent or temporary setting/configuration of the element A is configured/set to actually perform the operation B may be included. For example, if the element A is a general purpose processor, the processor may have a hardware configuration that can perform the operation B and be configured to actually perform the operation B by setting a permanent or temporary program (i.e., an instruction). If the element A is a dedicated processor, a dedicated arithmetic circuit, or the like, a circuit structure of the processor may be implemented so as to actually perform the operation B irrespective of whether the control instruction and the data are actually attached.

In the present specification (including the claims), if a term indicating inclusion or possession (e.g., “comprising”, “including”, or “having”) is used, the term is intended as an open-ended term, including inclusion or possession of an object other than a target object indicated by the object of the term. If the object of the term indicating inclusion or possession is an expression that does not specify a quantity or that suggests a singular number (i.e., an expression using “a” or “an” as an article), the expression should be interpreted as being not limited to a specified number.

In the present specification (including the claims), even if an expression such as “one or more” or “at least one” is used in a certain description, and an expression that does not specify a quantity or that suggests a singular number (i.e., an expression using “a” or “an” as an article) is used in another description, it is not intended that the latter expression indicates “one”. Generally, an expression that does not specify a quantity or that suggests a singular number (i.e., an expression using “a” or “an” as an article) should be interpreted as being not necessarily limited to a particular number.

In the present specification, if it is described that a particular advantage/result is obtained in a particular configuration included in an embodiment, unless there is a particular reason, it should be understood that that the advantage/result may be obtained in another embodiment or other embodiments including the configuration. It should be understood, however, that the presence or absence of the advantage/result generally depends on various factors, conditions, and/or states, and that the advantage/result is not necessarily obtained by the configuration. The advantage/result is merely an advantage/result that is obtained by the configuration described in the embodiment when various factors, conditions, and/or states are satisfied, and is not necessarily obtained in the invention according to the claim that defines the configuration or a similar configuration.

In the present specification (including the claims), if a term such as “maximize” or “maximization” is used, it should be interpreted as appropriate according to a context in which the term is used, including obtaining a global maximum value, obtaining an approximate global maximum value, obtaining a local maximum value, and obtaining an approximate local maximum value. It also includes obtaining approximate values of these maximum values, stochastically or heuristically. Similarly, if a term such as “minimize” or “minimization” is used, it should be interpreted as appropriate, according to a context in which the term is used, including obtaining a global minimum value, obtaining an approximate global minimum value, obtaining a local minimum value, and obtaining an approximate local minimum value. It also includes obtaining approximate values of these minimum values, stochastically or heuristically. Similarly, if a term such as “optimize” or “optimization” is used, the term should be interpreted as appropriate, according to a context in which the term is used, including obtaining a global optimum value, obtaining an approximate global optimum value, obtaining a local optimum value, and obtaining an approximate local optimum value. It also includes obtaining approximate values of these optimum values, stochastically or heuristically.

In the present specification (including the claims), if multiple hardware performs predetermined processes, each of the hardware may cooperate to perform the predetermined processes, or some of the hardware may perform all of the predetermined processes. Additionally, some of the hardware may perform some of the predetermined processes while another hardware may perform the remainder of the predetermined processes. In the present specification (including the claims), if an expression such as “one or more hardware perform a first process and the one or more hardware perform a second process” is used, the hardware that performs the first process may be the same as or different from the hardware that performs the second process. That is, the hardware that performs the first process and the hardware that performs the second process may be included in the one or more hardware. The hardware may include an electronic circuit, a device including an electronic circuit, or the like.

In the present specification (including the claims), if multiple storage devices (memories) store data, each of the multiple storage devices (memories) may store only a portion of the data or may store an entirety of the data. Additionally, a configuration in which some of the multiple storage devices store data may be included.

Although the embodiments of the present disclosure have been described in detail above, the present disclosure is not limited to the individual embodiments described above. Various additions, modifications, substitutions, partial deletions, and the like can be made without departing from the conceptual idea and spirit of the invention derived from the contents defined in the claims and the equivalents thereof. For example, in the embodiments described above, if numerical values or mathematical expressions are used for description, they are presented as an example and do not limit the scope of the present disclosure. Additionally, the order of respective operations in the embodiments is presented as an example and does not limit the scope of the present disclosure.

Regarding the above-described embodiment, the following supplementary notes are disclosed as one aspect and selective features of the invention.

### (Note 1)

An information processing apparatus comprising:
at least one memory; and
at least one processor,
wherein the at least one processor is configured to:
   determine, based on types of atoms to be analyzed by a second model, at least part of parameters of a first model having been trained with first data, and generate, by using the at least part of parameters of the first model, the second model different from the first model, the second model being a model from which an analysis result of an atomic structure is output by inputting the atomic structure.

### (Note 2)

The at least one processor is configured to train the second model by using second data.

### (Note 3)

The at least one processor is configured to generate, based on the types of atoms to be analyzed by the second model, the second data by using a third model different from the first model.

### (Note 4)

The first data is data generated by using the third model.

### (Note 5)

The third model is a neural network potential.

### (Note 6)

The first data and the second data each include atomic structures and analysis results of the atomic structures, and
the number of types of the atomic structures included in the first data is larger than the number of types of the atomic structures included in the second data.

### (Note 7)

The at least one processor is configured to acquire information about the types of atoms to be analyzed by the second model from another information processing apparatus.

### (Note 8)

The first model and the second model are models of the same type.

### (Note 9)

The first model and the second model each include parameters, and
the number of the parameters included in the second model is smaller than the number of the parameters included in the first model.

### (Note 10)

The type of the models is a moment tensor potential (MTP) model.

### (Note 11)

The number of types of atoms analyzable by the first model is larger than the number of types of atoms analyzable by the second model.

### (Note 12)

The at least one processor is configured to:
compare the analysis results output by the second model with verification data generated by a method different from a method using the first model and a method using the second model, and
output a comparison result as a numerical value.

### (Note 13)

The verification data is analysis results obtained by a third model having generated at least one of the first data or the second data.

### (Note 14)

The verification data is data obtained by experiment.

### (Note 15)

The analysis result of the atomic structure includes information about at least an energy or a force of the atomic structure.

### (Note 16)

The at least one processor is configured to train the first model using the first data before generating the second model.

### (Note 17)

An information processing system comprising:
a first information processing apparatus; and
a second information processing apparatus, wherein
the second information processing apparatus includes at least one processor and at least one memory,
the at least one processor of the second information processing apparatus is configured to transmit information including types of atoms to be analyzed by a second model to the first information processing apparatus, the second model being a model from which an analysis result of an atomic structure is output by inputting the atomic structure,
the first information processing apparatus includes at least one processor and at least one memory, and
the at least one processor of the first information processing apparatus is configured to:
   determine, based on the information received from the second information processing apparatus, at least part of parameters of a first model having been trained with first data, and
   generate, by using the at least part of parameters of the first model, the second model.

### (Note 18)

The first information processing apparatus is configured to train the second model by using second data.

### (Note 19)

The first information processing apparatus is configured to generate, based on the types of atoms to be analyzed by the second model, the second data by using a third model different from the first model.

### (Note 20)

The first data and the second data each include atomic structures and analysis results of the atomic structures, and
the number of types of the atomic structures included in the first data is larger than the number of types of the atomic structures included in the second data.

### (Note 21)

The first model and the second model each include parameters, and
the number of the parameters included in the second model is smaller than the number of the parameters included in the first model.

### (Note 22)

The analysis results of the atomic structures include information about at least energies or forces of the atomic structures.

### (Note 23)

A method implemented by at least one processor, the method comprising:
determining, based on types of atoms to be analyzed by a second model, at least part of parameters of a first model having been trained with first data; and
generating, by using the at least part of parameters of the first model, the second model different from the first model, the second model being a model from which an analysis result of an atomic structure is output by inputting the atomic structure.

What is claimed is:

1. An information processing apparatus comprising:
at least one memory; and
at least one processor,
wherein the at least one processor is configured to:
determine, based on types of atoms to be analyzed by a second model, at least part of parameters of a first model having been trained with first data, and
generate, by using the at least part of parameters of the first model, the second model different from the first model, the second model being a model from which an analysis result of an atomic structure is output by inputting the atomic structure.

2. The information processing apparatus according to claim 1, wherein the at least one processor is configured to train the second model by using second data.

3. The information processing apparatus according to claim 2, wherein the at least one processor is configured to generate, based on the types of atoms to be analyzed by the second model, the second data by using a third model different from the first model.

4. The information processing apparatus according to claim 3, wherein the first data is data generated by using the third model.

5. The information processing apparatus according to claim 3, wherein the third model is a neural network potential.

6. The information processing apparatus according to claim 2, wherein
the first data and the second data each include atomic structures and analysis results of the atomic structures, and
the number of types of the atomic structures included in the first data is larger than the number of types of the atomic structures included in the second data.

7. The information processing apparatus according to claim 1, wherein the at least one processor is configured to acquire information about the types of atoms to be analyzed by the second model from another information processing apparatus.

8. The information processing apparatus according to claim 1, wherein the first model and the second model are models of the same type.

9. The information processing apparatus according to claim 8, wherein
the first model and the second model each include parameters, and
the number of the parameters included in the second model is smaller than the number of the parameters included in the first model.

10. The information processing apparatus according to claim 8, wherein the type of the models is a moment tensor potential (MTP) model.

11. The information processing apparatus according to claim 1, wherein the number of types of atoms analyzable by the first model is larger than the number of types of atoms analyzable by the second model.

12. The information processing apparatus according to claim 2, wherein the at least one processor is configured to:
compare the analysis results output by the second model with verification data generated by a method different from a method using the first model and a method using the second model, and
output a comparison result as a numerical value.

13. The information processing apparatus according to claim 12, wherein the verification data is analysis results obtained by a third model having generated at least one of the first data or the second data.

14. The information processing apparatus according to claim 12, wherein the verification data is data obtained by experiment.

15. The information processing apparatus according to claim 1, wherein the analysis result of the atomic structure includes information about at least an energy or a force of the atomic structure.

16. The information processing apparatus according to claim 1, wherein the at least one processor is configured to train the first model using the first data before generating the second model.

17. An information processing system comprising:
a first information processing apparatus; and
a second information processing apparatus, wherein
the second information processing apparatus includes at least one processor and at least one memory,
the at least one processor of the second information processing apparatus is configured to transmit information including types of atoms to be analyzed by a second model to the first information processing apparatus, the second model being a model from which an analysis result of an atomic structure is output by inputting the atomic structure,
the first information processing apparatus includes at least one processor and at least one memory, and
the at least one processor of the first information processing apparatus is configured to:
determine, based on the information received from the second information processing apparatus, at least part of parameters of a first model having been trained with first data, and
generate, by using the at least part of parameters of the first model, the second model.

18. The information processing system according to claim 17, wherein the first information processing apparatus is configured to train the second model by using second data.

19. The information processing system according to claim 18, wherein the first information processing apparatus is configured to generate, based on the types of atoms to be analyzed by the second model, the second data by using a third model different from the first model.

20. A method implemented by at least one processor, the method comprising:
determining, based on types of atoms to be analyzed by a second model, at least part of parameters of a first model having been trained with first data; and
generating, by using the at least part of parameters of the first model, the second model different from the first model, the second model being a model from which an analysis result of an atomic structure is output by inputting the atomic structure.
